(19) United States
(12) Patent Application Publication
(10) Pub. No.: US 2026/0202117 A1
(43) Pub. Date: Jul. 16, 2026

(54) APPARATUS FOR STERILIZATION AND DEODORIZATION, AND REFRIGERATOR COMPRISING SAME

(71) Applicant: SAMSUNG ELECTRONICS CO., LTD., Suwon-si (KR)

(72) Inventors: Donghyo KIM, Suwon-si (KR); Myungju SHIN, Suwon-si (KR); Jinkook YOON, Suwon-si (KR); Bokyung LEE, Suwon-si (KR); Sanghoon LEE, Suwon-si (KR); Jisook LEE, Suwon-si (KR); Youngchul KO, Suwon-si (KR)

(73) Assignee: SAMSUNG ELECTRONICS CO., LTD., Suwon-si (KR)

(21) Appl. No.: 19/565,016

(22) Filed: Mar. 12, 2026

Related U.S. Application Data
(63) Continuation of application No. PCT/KR2024/015779, filed on Oct. 17, 2024.

(30) Foreign Application Priority Data
Nov. 29, 2023 (KR) 10-2023-0170183

(51) Int. Cl. F25D 17/04; A61L 9/20

(52) U.S. Cl. CPC F25D 17/042; A61L 9/20; A61L 2209/12; A61L 2209/14; A61L 2209/15; F25D 2317/0415; F25D 2317/0417

(57) ABSTRACT

The refrigerator comprises a cabinet, a storage chamber disposed inside the cabinet, a cold air duct disposed at a rear of the storage chamber, and a sterilizing and deodorizing device mounted inside the storage chamber, and to introduce air discharged from the cold air duct, sterilize and deodorize the introduced air, and discharge the sterilized and deodorized air into the storage chamber. The sterilizing and deodorizing device comprises a housing mounted in the storage chamber, a deodorizing filter deodorizing the air introduced into the housing, a sterilizing lamp to sterilize the air introduced into the housing, and having a cylindrical shape, and an insulating tape attached to a surface of the housing adjacent to the storage chamber, and comprising a reflective layer reflecting ultraviolet rays emitted from the sterilizing lamp and an insulating layer preventing heat generated from the sterilizing lamp from moving to the storage chamber.

### CROSS REFERENCE TO THE RELATED APPLICATION

This application is a continuation application, filed under 35 U.S.C. § 111(a), of International Application PCT/KR2024/015779 filed Oct. 17, 2024, and is based on and claims priority under 35 U.S.C. § 119 to Korean Patent Applications No. 10-2023-0170183, filed on Nov. 29, 2023, in the Korean Intellectual Property Office, the disclosures of which are incorporated by reference herein in their entireties.

### TECHNICAL FIELD

The present disclosure relates to a refrigerator including a sterilization and deodorization device having a heat dissipation and insulation structure applied thereto to prevent condensation that may occur due to heat generated from a sterilizing lamp.

### BACKGROUND ART

A refrigerator is an apparatus keeping food fresh by including a cabinet having a storage compartment and a cold air supply system for supplying cold air to the storage compartment. The storage compartment includes a refrigerating chamber for storing food in a refrigerated state by maintaining a temperature at about 0 to 5 degrees Celsius, and a freezing chamber for storing food in a frozen state by maintaining a temperature at about 0 to minus 30 degrees Celsius. A door may be provided on a front side of the cabinet to open and close the storage compartment. The door may be provided as a drawer-type door to open and close the storage compartment.

Horticultural products such as vegetables and fruits or agricultural and livestock products may be stored inside the storage compartment, and accordingly, these products may rot during distribution or storage in the storage compartment, leading to bacterial growth or foul odors. In this case, even if sources of the foul odors are removed, the odors may remain inside the storage compartment.

In order to remove bacteria or odors remaining inside the storage compartment, a sterilization and deodorization device capable of sterilizing and deodorizing air may be provided inside the storage compartment.

### DISCLOSURE

### Technical Problem

The present disclosure is directed to providing a refrigerator including a sterilization and deodorization device capable of preventing dew from forming on a wall surface of a housing adjacent to a storage compartment by attaching a thermal insulating tape to the wall surface of the housing adjacent to the storage compartment.

The present disclosure is also directed to providing a refrigerator including a sterilization and deodorization device capable of preventing loss of output of a sterilizing lamp by attaching a thermal insulating tape to a wall surface of a housing adjacent to a storage compartment.

The present disclosure is also directed to providing a refrigerator including a sterilization and deodorization device capable of extending the life of a sterilizing lamp by providing a heat dissipation support supporting the sterilizing lamp to dissipate heat generated from the sterilizing lamp.

The present disclosure is also directed to providing a refrigerator including a sterilization and deodorization device capable of preventing photodegradation of a housing by attaching an thermal insulating tape to a wall surface of the housing adjacent to a storage compartment.

The present disclosure is also directed to providing a refrigerator a sterilization and deodorization device in which a sterilizing lamp and a deodorizing filter are provided within one housing.

The present disclosure is also directed to providing a refrigerator including a sterilization and deodorization device in which a sterilizing lamp is disposed in a central portion inside a housing to sufficiently secure an air sterilization area and sterilization time.

The present disclosure is also directed to providing a refrigerator including a sterilization and deodorization device capable of simultaneously securing sterilization and/or deodorization of air using a sterilizing lamp by disposing the sterilizing lamp between an inlet and a deodorizing filter.

The present disclosure is also directed to providing a refrigerator including a sterilization and deodorization device capable of sterilizing and/or deodorizing air using a sterilizing lamp, thereby improving power consumption efficiency.

The present disclosure is also directed to providing a refrigerator including a sterilization and deodorization device capable of minimizing loss of air flow during sterilization and/or deodorization of air by implementing a straight flow path in which air introduced into an inlet flows along the inlet, a sterilizing lamp, a deodorizing filter, and an outlet.

The present disclosure is also directed to providing a refrigerator including a sterilization and deodorization device whose volume may be minimized by arranging a distance between the inlet and the sterilizing lamp to be longer than a distance between the sterilizing lamp and the deodorizing filter.

The present disclosure is also directed to providing a refrigerator including a sterilization and deodorization device capable of preventing ultraviolet rays radiated from a sterilizing lamp from leaking out of a housing through an inlet and a discharge port by installing a rib in the housing.

Technical tasks to be achieved in the present disclosure are not limited to the technical tasks mentioned above, and other technical tasks not mentioned will be clearly understood by those skilled in the art from the description below.

### Technical Solution

A refrigerator according to an embodiment of the present disclosure may include a cabinet, a storage compartment provided inside the cabinet, a cold air duct disposed at the rear of the storage compartment, and a sterilization and deodorization device mounted inside the storage compartment and configured to introduce air discharged from the cold air duct, sterilize and deodorize the air, and then discharge the air into the storage compartment. The sterilization and deodorization device may include a housing mounted in the storage compartment, a deodorizing filter provided to deodorize air introduced into the housing, a sterilizing lamp provided to sterilize air introduced into the housing and having a cylindrical shape, and an thermal insulating tape attached to a wall surface of the housing adjacent to the storage compartment and including a reflective layer reflecting ultraviolet rays radiated from the sterilizing lamp and an thermal insulating layer preventing heat generated from the sterilizing lamp from being transferred to the storage compartment.

A refrigerator may comprise a cabinet, a storage compartment disposed inside the cabinet, a cold air duct disposed at a rear of the storage compartment, and a sterilization and deodorization device mounted inside the storage compartment, connected to the cold air duct, and configured to introduce air discharged from the cold air duct, sterilize and deodorize the introduced air, and discharge the sterilized and deodorized air into the storage compartment.

The sterilization and deodorization device may comprise a housing mounted in the storage compartment, a deodorizing filter configured to deodorize the air introduced into the housing, a sterilizing lamp configured to sterilize the air introduced into the housing, the sterilizing lamp having a cylindrical shape, and a thermal insulating tape attached to a surface of the housing adjacent to the storage compartment.

The thermal insulating tape may comprise a reflective layer configured to reflect ultraviolet rays radiated from the sterilizing lamp; and a thermal insulating layer configured to prevent heat generated from the sterilizing lamp from being transferred to the storage compartment.

The housing may include a first housing mounted on an upper wall of the storage compartment and a second housing mounted on a lower portion of the first housing, and the surface of the housing is an inner surface of the second housing where the thermal insulating tape is attached to.

The reflective layer may be formed of a metallic material.

The sterilization and deodorization device may further comprise a heat dissipation support configured to support the sterilizing lamp and dissipate heat generated from the sterilizing lamp.

The sterilization and deodorization device may further comprise a plurality of the heat dissipation supports that is made of a metallic material.

The heat dissipation support may comprise a contact part configured to be in contact with the sterilizing lamp, and a support part connected to the contact part and fixed to the surface of the housing adjacent to the storage compartment to support the sterilizing lamp.

The support part may be disposed such that at least a portion thereof is in contact with the surface of the housing adjacent to the storage compartment.

The heat dissipation support may further comprise an extension part extending along the surface of the housing from the support part.

The extension part may extend in a direction parallel to a flow direction of the air that is introduced into the sterilization and deodorization device.

The extension part may have a same width as the support part.

The extension part may comprise an expansion part has a width wider than the width of the support part.

The expansion may be connected to each other in a direction perpendicular to the flow direction of the air that is introduced into the sterilization and deodorization device.

The sterilization and deodorization device may further comprise an inlet into which the air discharged from the cold air duct is introduced.

The extension part may extend from the support part to the inlet.

The thermal insulating tape may be attached to the surface of the housing to cover an upper portion of the extension part.

The sterilization and deodorization device may further comprise an inlet into which the air discharged from the cold air duct is introduced, and the housing includes a blocking rib to prevent the ultraviolet rays from leaking into the cold air duct through the inlet.

The blocking rib may include a first blocking rib and a second blocking rib which is separated apart from the blocking rib in a flow direction of the air that is introduced into the sterilization and deodorization device.

A sterilization and deodorization device according to an embodiment of the present disclosure, which introduces air discharged from a cold air duct disposed at the rear of a storage compartment, sterilizes and deodorizes the air, and then discharges the air into the storage compartment, may include a housing including a first housing mounted on an upper wall of the storage compartment and a second housing mounted on a lower portion of the first housing, an inlet provided in the housing to allow air discharged from the cold air duct to be introduced into the housing, a deodorizing filter provided to deodorize air introduced into the inlet, a sterilizing lamp provided to sterilize air introduced into the inlet and having a cylindrical shape, a heat dissipation support provided to support the sterilizing lamp and dissipate heat generated from the sterilizing lamp, and a thermal insulating tape provided to be attached to a lower wall of the second housing and including a reflective layer reflecting ultraviolet rays radiated from the sterilizing lamp and a thermal insulating layer preventing heat generated from the sterilizing lamp from being transferring to the storage compartment.

### DESCRIPTION OF DRAWINGS

FIG. 1 is a view illustrating a refrigerator with the door opened according to an embodiment.

FIG. 2 is a schematic cross-sectional side view of the refrigerator according to an embodiment.

FIG. 3 is a cross-sectional view illustrating that a sterilization and deodorization device mounted on an upper wall of a refrigerating chamber is connected to a cold air duct according to an embodiment.

FIG. 4 is a view illustrating that the sterilization and deodorization device is mounted on the upper wall of the refrigerating chamber according to an embodiment.

FIG. 5 is a view illustrating a state in which the sterilization and deodorization device is disassembled from the upper wall of the refrigerating chamber according to an embodiment.

FIG. 6 is a view illustrating the sterilization and deodorization device according to an embodiment.

FIG. 7 is a view illustrating a state in which the sterilization and deodorization device is disassembled according to an embodiment.

FIG. 8 is a plan view illustrating that a sterilizing lamp, a deodorizing filter, and a deodorizing lamp are mounted and a thermal insulating tape is attached, in the inside of the second housing according to an embodiment.

FIG. 9 is a cross-sectional view illustrating that a blocking rib is provided in a portion adjacent to an inlet of the sterilization and deodorization device according to an embodiment.

FIG. 10 is a cross-sectional view illustrating that a first blocking rib provided in a portion adjacent to an inlet of the sterilization and deodorization device is formed further in front than a second blocking rib according to an embodiment.

FIG. 11 is a cross-sectional view illustrating that a second blocking rib provided in a portion adjacent to an inlet of the sterilization and deodorization device is formed further in front than a first blocking rib according to an embodiment.

FIG. 12 is a view illustrating a state in which the sterilization and deodorization device including a heat dissipation support supporting the sterilizing lamp is exploded according to an embodiment.

FIG. 13 is a view illustrating that the heat dissipation support supports the sterilizing lamp according to an embodiment.

FIG. 14 is a schematic cross-sectional view of the sterilization and deodorization device including the heat dissipation support supporting the sterilizing lamp according to an embodiment.

FIG. 15 is a plan view illustrating that the sterilizing lamp, the deodorizing filter, and the deodorizing lamp are mounted, the heat dissipation support supports the sterilizing lamp, and the thermal insulating tape is attached, in the inside of the second housing according to an embodiment.

FIG. 16 is a view illustrating that the heat dissipation support supporting the sterilizing lamp includes an extension part extending from a support part according to an embodiment.

FIG. 17 is a schematic cross-sectional view of the sterilization and deodorization device in which the heat dissipation support includes the extension part according to an embodiment.

FIG. 18 is a plan view illustrating that the sterilizing lamp, the deodorizing filter, and the deodorizing lamp are mounted, the heat dissipation support including the extension part supports the sterilizing lamp, and the thermal insulating tape is attached, in the inside of the second housing according to an embodiment.

FIG. 19 is a view illustrating that the heat dissipation support includes an expansion part having a width wider than a width of the support part according to an embodiment.

FIG. 20 is a schematic cross-sectional view of the sterilization and deodorization device in which the heat dissipation support includes the expansion part according to an embodiment.

FIG. 21 is a plan view illustrating that the sterilizing lamp, the deodorizing filter, and the deodorizing lamp are mounted, the heat dissipation support including the expansion part supports the sterilizing lamp, and the thermal insulating tape is attached, in the inside of the second housing according to an embodiment.

FIG. 22 is a view illustrating that expansion parts of the heat dissipation support are connected to each other in a direction perpendicular to a flow direction of air according to an embodiment.

FIG. 23 is a plan view illustrating that the sterilizing lamp, the deodorizing filter, and the deodorizing lamp are mounted, the heat dissipation support including the expansion parts connected to each other in a direction perpendicular to the flow direction of air supports the sterilizing lamp, and the thermal insulating tape is attached, in the inside of the second housing according to an embodiment.

### MODE OF THE DISCLOSURE

Various embodiments of the present disclosure and the terms used therein are not intended to limit the technical features described in the present disclosure to specific embodiments, and should be understood to include various modifications, equivalents, or substitutes of the embodiments.

In connection with the explanation of the drawings, like reference numbers may be used for like or related components.

The singular form of a noun corresponding to an item may include one item or a plurality of items, unless the relevant context clearly dictates otherwise.

In the present disclosure, each of phrases such as “A or B,” “at least one of A and B,” “at least one of A or B,” “A, B or C,” “at least one of A, B and C,” and “at least one of A, B, or C” may include any one of the items listed together in the corresponding one of the phrases, or all possible combinations thereof.

The term “and/or” includes any combination of a plurality of related components or any one of a plurality of related components.

Terms such as “first,” “second,” “primary,” and “secondary” may simply be used to distinguish a given component from other corresponding components, and do not limit the corresponding components in any other aspect (e.g., importance or order).

The terms “front surface,” “rear surface,” “upper surface,” “lower surface,” “side surface,” “left side,” “right side,” “upper portion,” “lower portion,” and the like used in the present disclosure are defined based on the drawings, and the shape and position of each component are not limited by these terms.

The terms “includes” and “has” are intended to indicate that there are features, numbers, steps, operations, components, parts, or combinations thereof described in the present disclosure, and do not exclude the presence or addition of one or more other features, numbers, steps, operations, components, parts, or combinations thereof.

When any component is referred to as being “connected to”, “coupled to,” “supported by” or “in contact with” another component, this includes a case in which the components are indirectly connected to, coupled to, supported by, or in contact with each other through a third component as well as directly connected to, coupled to, supported by, or in contact with each other.

When any component is referred to as being located “on” or “above” another component, this includes not only a case in which any component is in contact with another component but also a case in which another component is present between the two components.

A refrigerator according to an embodiment may include a cabinet.

The “cabinet” may include an inner case, an outer case disposed on the outside of the inner case, and an insulator provided between the inner case and the outer case.

The “inner case” may include at least one of a case, a plate, a panel and a liner forming a storage compartment. The inner case may be formed as a single body or may be formed by assembling a plurality of plates. The “outer case” may form an outer appearance of the cabinet and may be coupled to the outside of the inner case so that the insulator is disposed between the inner case and the outer case.

The “insulator” may insulate the inside and outside of the storage compartment so that a temperature inside the storage compartment may be maintained at a set appropriate temperature without being affected by an external environment of the storage compartment. According to an embodiment, the insulator may include a foam insulator such as polyurethane foam. A foam insulator may be formed by injecting and foaming urethane foam mixed with polyurethane and foaming agent between the inner case and the outer case.

According to an embodiment, the insulator may further include a vacuum insulator in addition to the foam insulator, or may be configured as only the vacuum insulator instead of the foam insulator. The vacuum insulator may include a core material and an outer shell material that accommodates the core material and seals the inside thereof with a vacuum or a pressure close to vacuum. However, the insulator is not limited to the foam insulator or vacuum insulator described above and may include various materials that may be used for insulation.

The “storage compartment” may include a space defined by the inner case. The storage compartment may further include an inner case defining a space corresponding to the storage compartment. The storage compartment may store various items such as food, medicine, and cosmetics, and may be formed such that at least one side thereof is open to allow items to be put in and to be taken out.

The refrigerator may include one or more storage compartments. When two or more storage compartments are formed in the refrigerator, the respective storage compartments may have different uses and may be maintained at different temperatures. To this end, the respective storage compartments may be partitioned from each other by partitions including the insulators.

The storage compartment may be provided to be maintained at an appropriate temperature range depending on the use, and may include a “refrigerating chamber,” a “freezing chamber,” or a “variable temperature chamber” depending on the use and/or temperature range. The refrigerating chamber may be maintained at an appropriate temperature for storing items in a refrigerated state, and the freezing chamber may be maintained at an appropriate temperature for storing items in a frozen state. “Refrigerating” may refer to cooling items to the point where the items are not frozen, and as an example, the refrigerating chamber may be maintained in a temperature ranging from zero degree Celsius to seven degrees Celsius. “Freezing” may refer to cooling items such that the items are freezing or maintained in a frozen state, as an example, the freezing chamber may be maintained at a temperature ranging from minus twenty degrees Celsius to minus one degree Celsius. The variable temperature chamber may be used as any one of the refrigerating chamber and the freezing chamber, depending on a selection of a user or regardless of the selection of the user.

In addition to names such as “refrigerating chamber,” “freezing chamber,” and “variable temperature chamber,” the storage compartment may be referred to as various names such as “vegetable chamber,” “fresh chamber,” “cooling chamber,” and “ice making chamber,” terms such as “refrigerating chamber,” “freezing chamber,” and “variable temperature chamber” used below should be understood to encompass storage compartments with corresponding uses and temperature ranges, respectively.

According to an embodiment, the refrigerator may include at least one door configured to open and close the one open side of the storage compartment. The doors may each be provided to open and close the one or more storage compartments, or the one door may be provided to open and close a plurality of the storage compartments. The door may be rotatably or slidably installed on a front side of the cabinet.

The “door” may be configured to seal the storage compartment when closed. Like the cabinet, the door may include the insulator to insulate the storage compartment when closed.

According to an embodiment, the door may include a door outer plate forming a front surface of the door, a door inner plate forming a rear surface of the door and facing the storage compartment, an upper cap, a lower cap, and a door insulator provided inside the upper and lower caps.

Edges of the door inner plate may be provided with a gasket sealing the storage compartment by coming into close contact with the front side of the cabinet when the door is closed. The door inner plate may include a dyke protruding rearward so that a door basket for storing items is mounted.

According to an embodiment, the door may include a door body, and a front panel detachably coupled to a front side of the door body and forming the front surface of the door. The door body may include the door outer plate forming a front surface of the door body, the door inner plate forming a rear surface of the door body and facing the storage compartment, the upper cap, the lower cap, and the door insulator provided inside the upper and lower caps.

Refrigerators may be classified into a French door type, a side-by-side type, a bottom mounted freezer (BMF) type, a top mounted freezer (TMF) type, and a one-door refrigerator type depending on the arrangement of doors and storage compartments.

According to an embodiment, the refrigerator may include a cold air supply device configured to supply cold air to the storage compartment.

The “cold air supply device” may include a machine, mechanism, electronic device, and/or a system combining them capable of generating cold air and guiding the cold air to cool the storage compartment.

According to an embodiment, the cold air supply device may generate cold air through a refrigeration cycle including compression, condensation, expansion, and evaporation processes of a refrigerant. To this end, the cold air supply device may include a refrigeration cycle device having a compressor, a condenser, an expansion device, and an evaporator capable of driving the refrigeration cycle. According to an embodiment, the cold air supply device may include a semiconductor such as a thermoelectric element. The thermoelectric element may cool the storage compartment by generating heat and cooling through the Peltier effect.

According to an embodiment, the refrigerator may include a machine room in which at least some components belonging to the cold air supply device are disposed.

The “machine room” may be provided to be partitioned and insulated from the storage compartment in order to prevent heat generated from the components disposed in the machine room from being transferred to the storage compartment. The inside of the machine room may be configured to communicate with the outside of the cabinet to dissipate heat from the components disposed inside the machine room.

According to an embodiment, the refrigerator may include a dispenser provided on the door to provide water and/or ice. The dispenser may be provided on the door such that the user may access the door without opening the door.

According to an embodiment, the refrigerator may include an ice making device provided to produce ice. The ice making device may include an ice making tray provided to store water, an ice moving device provided to separate the ice from the ice making tray, and an ice bucket provided to store the ice produced in the ice making tray.

According to an embodiment, the refrigerator may include a controller configured to control the refrigerator.

The “controller” may include memory provided to store or remember programs and/or data for controlling the refrigerator, and a processor provided to output a control signal for controlling the cold air supply device and the like according to the programs and/or data stored in the memory.

The memory stores or records a variety of information, data, commands, programs, and the like required for operations of the refrigerator. The memory may remember temporary data generated while generating control signals for controlling components included in the refrigerator. The memory may include at least one of volatile memory and non-volatile memory, or a combination thereof.

The processor controls the overall operation of the refrigerator. The processor may control the components of the refrigerator by executing the programs stored in the memory. The processor may include a separate NPU to perform operations of an artificial intelligence model. The processor may also include a central processor, a graphics processor (GPU), and the like. The processor may generate a control signal for controlling an operation of the cold air supply device. For example, the processor may receive temperature information of the storage compartment from a temperature sensor, and generate a cooling control signal for controlling the operation of the cold air supply device based on the temperature information of the storage compartment.

Additionally, the processor may process user input of a user interface according to the programs and/or data memorized/stored in the memory and control an operation of the user interface. The user interface may be provided using an input interface and an output interface. The processor may receive the user input from the user interface. The processor may also transmit a display control signal and image data for displaying an image on the user interface to the user interface in response to the user input.

The processor and the memory may be provided integrally or may be provided separately. The processor may include one or more processors. For example, the processor may include a main processor and at least one sub-processor. The memory may include one or more memories.

According to an embodiment, the refrigerator may include a processor and a memory to control all the components included in the refrigerator, and may include a plurality of processors and a plurality of memories to individually control the components of the refrigerator. For example, the refrigerator may include a processor and memory to control the operation of the cold air supply device depending on output of the temperature sensor. Also, the refrigerator may be separately equipped with a processor and memory to control the operation of the user interface according to user input.

A communication module may communicate with an external device such as a server, a mobile device, and another home appliance through a nearby access point (AP). The access point (AP) may connect a local area network (LAN) to which the refrigerator or a user device is connected to a wide area network (WAN) to which the server is connected. The refrigerator or the user device may be connected to the server via the wide area network (WAN).

The input interface may include a key, a touch screen, a microphone, and the like. The input interface may receive user input and transmit the user input to the processor.

The output interface may include a display, a speaker, and the like. The output interface may output various notifications, messages, a variety of information, and the like generated by the processor.

Hereinafter, embodiments of the present disclosure will be described in detail with reference to the accompanying drawings.

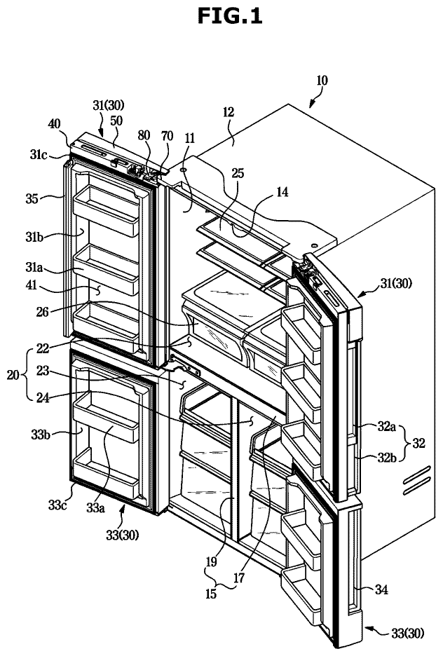

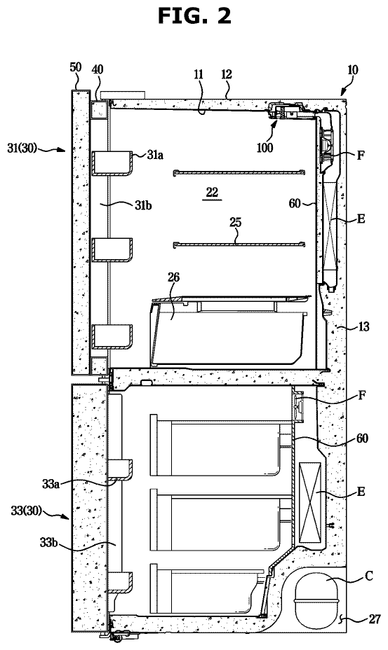

FIG. 1 is a view illustrating a refrigerator with the door opened according to an embodiment. FIG. 2 is a schematic cross-sectional side view of the refrigerator according to an embodiment.

As illustrated in FIGS. 1 and 2, a refrigerator may include a cabinet 10, a storage compartment 20 formed by partitioning upper and lower portions inside cabinet 10, a door 30 configured to open and close the storage compartment 20, and a cold air supply device (not shown) for supplying cold air to the storage compartment 20.

The cabinet 10 may be configured to include an inner case 11 forming the storage compartment 20, an outer case 12 coupled to the outside of the inner case 11 to form an outer appearance, and an insulator 13 formed by being foamed between the inner case 11 and the outer case 12 to insulate the storage compartment 20.

At a lower rear side of the cabinet 10, a compressor C for compressing refrigerant and a machine room 27 in which a condenser (not shown) for condensing the refrigerant compressed by the compressor C is installed may be provided.

The cold air supply device may include the compressor C installed in the machine room 27 to compress the refrigerant, the condenser (not shown) installed in the machine room 27 to condense the refrigerant, an expansion valve (not shown) for expanding the refrigerant condensed by the condenser, an evaporator E installed at the rear of the storage compartment 20 to generate cold air, a fan F for inducing cold air generated in the evaporator E to be discharged to the storage compartment 20, and a cold air duct 60 installed at the rear of the storage compartment 20 to discharge cold air induced by the fan F to the storage compartment 20. The evaporator E, fan F, and cold air duct 60, which are disposed at the rear of the storage compartment 20, may be disposed at the rear of a refrigerating chamber 22 and freezing chambers 23 and 24, respectively.

The storage compartment 20 may be partitioned in plurality by a partition 15, and pluralities of shelves 25 and storage containers 26 may be provided inside the storage compartment 20 so that food and the like may be stored.

The storage compartment 20 may be divided into a plurality of the storage chambers 22, 23, and 24 by the partition 15, and the partition 15 may include a first partition 17 horizontally coupled to the inside of the storage compartment 20 to divide the storage compartment 20 into the upper storage chamber 22 and the lower storage chambers 23 and 24, and a second partition 19 vertically coupled to the lower storage chambers 23 and 24 to divide the lower storage chambers 23 and 24 into the first storage chamber 23 and the second storage chamber 24.

The partition 15 having a T shape by coupling the first partition 17 and the second partition 19 may divide the storage compartment 20 into three spaces. Among the upper storage chamber 22 and the lower storage chambers 23 and 24 divided by the first partition 17, the upper storage chamber 22 may be used as a refrigerating chamber, and the lower storage chambers 23 and 24 may be used as a freezing chamber.

The lower storage chambers 23 and 24 may be used entirely as freezing chambers, but the first storage chamber 23 may be used as a freezing chamber and the second storage chamber 24 may be used as a refrigerating chamber, and the first storage chamber 23 may be used as a freezing chamber and the second storage chamber 24 may be used as both a freezing chamber and a refrigerating chamber.

The division of the storage compartment 20 as described above is an example only, and each of the storage chambers 22, 23, and 24 may be used differently from the above use.

The refrigerating chamber 22 and the freezing chambers 23 and 24 may each be opened and closed by the door 30 rotatably coupled to the cabinet 10.

The door 30 may include a pair of refrigerating chamber doors 31 rotatably coupled to the cabinet 10 to open and close the refrigerating chamber 22, and a pair of freezing chamber doors 33 rotatably coupled to the cabinet 10 to open and close the freezing chambers 23 and 24.

The pair of refrigerating chamber doors 31 may be opened and closed, respectively, through a pair of refrigerating chamber door handles 32 including a first door handle 32a or a second door handle 32b. The refrigerating chamber 22 may be opened and closed by the pair of refrigerating chamber doors 31, and a rotating bar 35 may be provided on at least one of the pair of refrigerating chamber doors 31 so that the pair of refrigerating chamber doors 31 may be sealed without a gap being created between the pair of refrigerating chamber doors 31 when the pair of refrigerating chamber doors 31 is closed. The rotating bar 35 may be rotatably coupled to at least one of the pair of refrigerating chamber doors 31. The rotating bar 35 may be guided to rotate according to the opening and closing of the refrigerating chamber door 31 by the rotating guide 14 formed in the cabinet 10.

The pair of freezing chamber doors 33 may each be opened and closed by a freezing chamber door handle 34. Sliding doors may be applied to the doors for opening and closing the freezing chambers 23 and 24.

Door shelves 31a and 33a capable of storing food may be provided on rear surfaces of the pair of refrigerating chamber doors 31 and the pair of freezing chamber doors 33, respectively.

Each of the door shelves 31a and 33a may include each of shelf supports 31b and 33b formed to extend vertically from each of the doors 31 and 33 so as to support each of the door shelves 31a and 33a on left and right sides of each of the door shelves 31a and 33a. The shelf supports 31b and 33b may be provided to extend from the doors 31 and 33, respectively. The shelf supports 31b and 33b may be detachably provided on the doors 31 and 33, respectively, as separate components.

Additionally, each of first gaskets 31c and 33c may be provided on a rear edge of each of the doors 31 and 33 to seal a gap between each of the doors 31 and 33 and the cabinet 10 in a state in which each of the doors 31 and 33 is closed. Each of the first gaskets 31c and 33c may be installed in a loop shape along the edge on the rear surface of each of the doors 31 and 33, and may include a magnet (not shown) therein.

The refrigerating chamber door 31 may be provided as a double door including a first door 40 and a second door 50.

The first door 40 may be rotatably connected to the cabinet 10 by a first hinge 70 and may open and close the refrigerating chamber 22. The door shelf 31a, shelf support 31b, and first gasket 31c described above may be provided on the first door 40.

The first door 40 may include an opening 41 formed to allow a user to access the door shelf 31a to put or take food in or out in a state in which the first door 40 is closed. The opening 41 may be formed by penetrating the first door 40 and may be opened and closed by the second door 50.

The second door 50 may be provided in front of the first door 40 to be capable of opening and closing the opening 41 of the first door 40, and may be provided to be rotatable in the same direction as the first door 40. The drawing illustrates that the second door 50 is rotatable relative to the first door 40 by being rotatably supported on a second hinge 80 installed on the first door 40, but the present disclosure is not limited thereto, and the second door 50 may be provided to be rotatable relative to the cabinet 10 by the second hinge 80 being installed on the cabinet 10.

The second door 50 may include a second gasket (not shown) to maintain sealing with the first door 40. The second gasket may be installed in a loop shape along an edge of a rear surface of the second door 50, and may include a magnet (not shown) therein.

A sterilization and deodorization device 100 configured to sterilize and deodorize air may be mounted inside the storage compartment 20. The sterilization and deodorization device 100 may be mounted inside the refrigerating chamber 22. The sterilization and deodorization device 100 may be mounted on an upper wall of the refrigerating chamber 22. A detailed description of the sterilization and deodorization device 100 will be provided later.

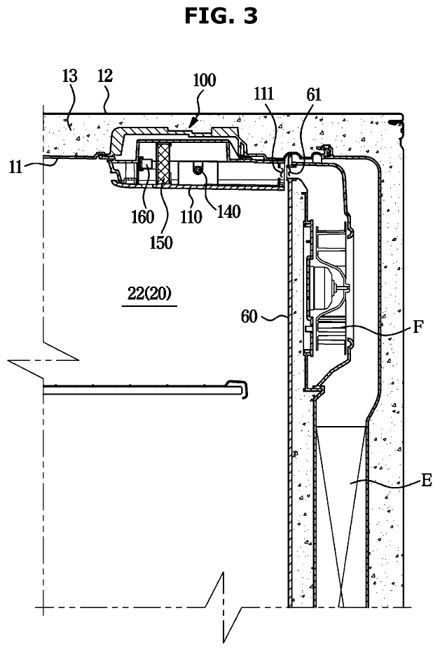

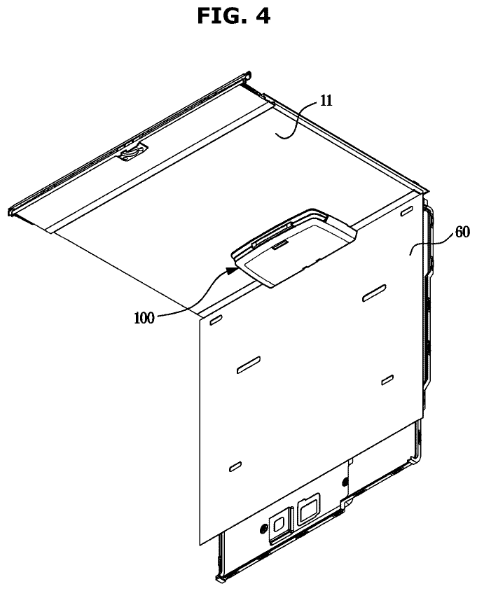

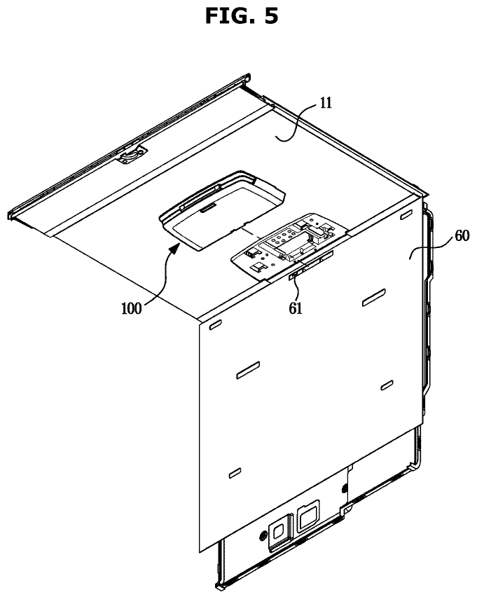

FIG. 3 is a cross-sectional view illustrating that a sterilization and deodorization device mounted on an upper wall of a refrigerating chamber is connected to a cold air duct according to an embodiment. FIG. 4 is a view illustrating that the sterilization and deodorization device is mounted on the upper wall of the refrigerating chamber according to an embodiment. FIG. 5 is a view illustrating a state in which the sterilization and deodorization device is disassembled from the upper wall of the refrigerating chamber according to an embodiment.

As illustrated in FIGS. 3 to 5, the sterilization and deodorization device 100 may be mounted on the upper wall of the refrigerating chamber 22. The sterilization and deodorization device 100 may include a housing 110 mounted on the upper wall of the refrigerating chamber 22. A detailed description of the housing 110 will be provided later.

The sterilization and deodorization device 100 mounted on the upper wall of the refrigerating chamber 22 may be connected to the cold air duct 60. The cold air duct 60 may be disposed at the rear of the refrigerating chamber 22. The cold air generated in the evaporator E may be induced to move along the cold air duct 60 by the fan F. The cold air moved along the cold air duct 60 by the fan F may be discharged from the cold air duct 60 through a cold air duct discharge port 61. The cold air discharged from the cold air duct discharge port 61 may be introduced into the sterilization and deodorization device 100. The cold air duct discharge port 61 may be connected to an inlet 111 of the sterilization and deodorization device 100. Accordingly, the cold air discharged through the cold air duct discharge port 61 may be introduced into the sterilization and deodorization device 100 through the inlet 111. Hereinafter, the cold air discharged from the cold air duct discharge port 61 and introduced into the inlet 111 will be referred to as air.

The air introduced into the sterilization and deodorization device 100 through the inlet 111 may be sterilized by a sterilizing lamp 140 mounted inside the sterilization and deodorization device 100. The air introduced into the sterilization and deodorization device 100 through the inlet 111 may be deodorized by a deodorizing filter 150 mounted inside the sterilization and deodorization device 100. The air sterilized and deodorized by being introduced into the sterilization and deodorization device 100 may be discharged into the refrigerating chamber 22 through a discharge port 170. A detailed configuration of the sterilization and deodorization device 100 will be provided later.

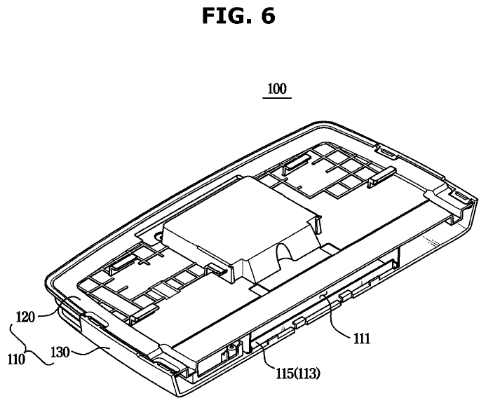

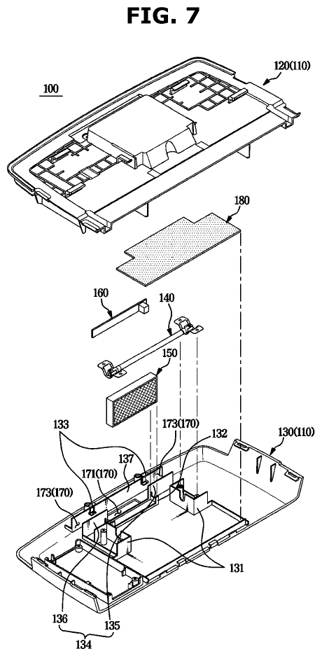

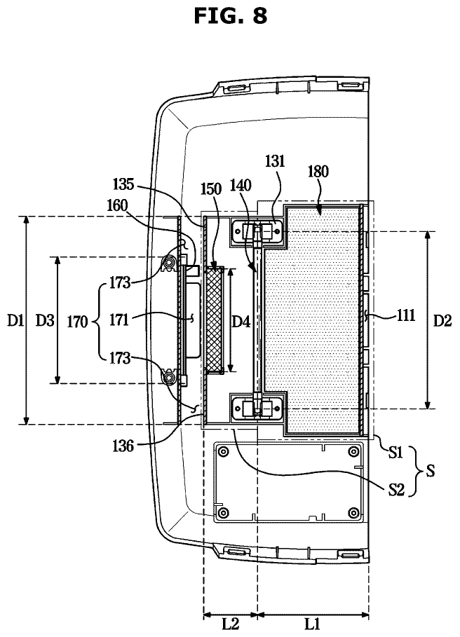

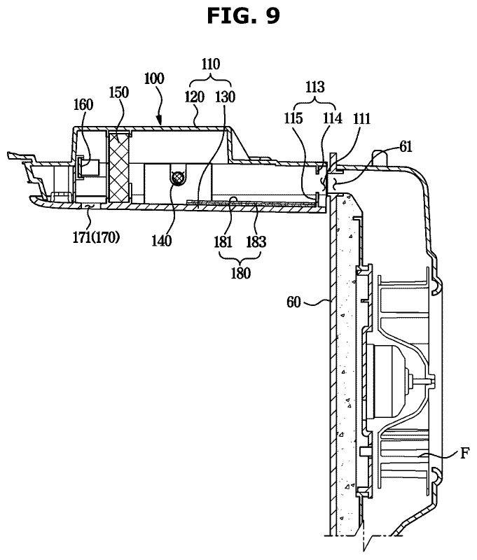

FIG. 6 is a view illustrating the sterilization and deodorization device according to an embodiment. FIG. 7 is a view illustrating a state in which the sterilization and deodorization device is disassembled according to an embodiment. FIG. 8 is a plan view illustrating that a sterilizing lamp, a deodorizing filter, and a deodorizing lamp are mounted and a thermal insulating tape is attached, in the inside of the second housing according to an embodiment. FIG. 9 is a cross-sectional view illustrating that a blocking rib is provided in a portion adjacent to an inlet of the sterilization and deodorization device according to an embodiment.

Hereinafter, when the sterilization and deodorization device 100 is described, a portion where the discharge port 170 is located may be referred to as the front, and a portion where the inlet 111 is located may referred to as the rear, based on the inlet 111 and the discharge port 170. Although the sterilization and deodorization device 100 is described as being mounted on the upper wall of the refrigerating chamber 22, the present disclosure is not limited thereto. That is, the sterilization and deodorization device 100 may be mounted on a part other than the upper wall of the refrigerating chamber 22. Also, the sterilization and deodorization device 100 may be mounted inside the freezing chambers 23 and 24 rather than the refrigerating chamber 22. Also, the sterilization and deodorization device 100 may be mounted inside all of the refrigerating chamber 22 and the freezing chambers 23 and 24 (see FIG. 2).

As illustrated in FIGS. 6 to 9, the sterilization and deodorization device 100 may include the housing 110. The housing 110 may be mounted on the upper wall of the refrigerating chamber 22. The housing 110 may include a first housing 120 mounted on the upper wall of the refrigerating chamber 22. The housing 110 may include a second housing 130 mounted on a lower portion of the first housing 120. The housing 110 may include the inlet 111 through which air is introduced. The inlet 111 may be formed by the first housing 120 and the second housing 130. The inlet 111 may be connected to the cold air duct discharge port 61 of the cold air duct 60. Air discharged from the cold air duct discharge port 61 of the cold air duct 60 may be introduced into the inlet 111. Air introduced into the housing 110 through the inlet 111 may be sterilized and deodorized by the sterilizing lamp 140 and deodorizing filter 150 mounted inside the housing 110. The sterilized and deodorized air may be discharged into the refrigerating chamber 22 through the discharge port 170 (see FIGS. 2 and 3).

The housing 110 may include a blocking rib 113 preventing ultraviolet rays radiated from the sterilizing lamp 140 from leaking out of the housing 110 through the inlet 111. Air introduced into the housing 110 may be sterilized by ultraviolet rays radiated from the sterilizing lamp 140 mounted inside the housing 110. The ultraviolet rays radiated from the sterilizing lamp 140 may be prevented from leaking into the cold air duct 60 through the inlet 111 by the blocking rib 113.

The blocking rib 113 may be provided in each of the first housing 120 and the second housing 130 adjacent to the inlet 111. The blocking rib 113 may include a first blocking rib 114 formed in the first housing 120. The first blocking rib 114 may be formed to extend downward from an upper wall of the first housing 120.

The blocking rib 113 may include a second blocking rib 115 formed in the second housing 130. The second blocking rib 115 may be formed to extend upward from a lower wall of the second housing 130.

The first blocking rib 114 and the second blocking rib 115 may be formed at the same position on basis of a flow direction of air that is introduced into the inlet 111. Accordingly, a space may be formed between the first blocking rib 114 and the second blocking rib 115 so that air introduced into the inlet 111 may flow into the inside of the housing 110. That is, the first block rib 114 and the second block rib 115 may be spaced apart from each other.

A part of the ultraviolet rays radiated from the sterilizing lamp 140 may be prevented from leaking into the inlet 111 by the blocking rib 113, but because the space is formed between the first blocking rib 114 and the second blocking rib 115, the part may leak out of the inlet 111. The ultraviolet rays leaked out of the inlet 111 through the space between the first blocking rib 114 and the second blocking rib 115 may be introduced into the cold air duct 60 through the cold air duct discharge port 61. A portion of the cold air duct 60 adjacent to the cold air duct discharge port 61 may be sterilized by the ultraviolet rays introduced into the cold air duct 60 through the cold air duct discharge port 61.

The second housing 130 may include a sterilizing lamp mounting part 131 on which the sterilizing lamp 140 is mounted. The sterilizing lamp mounting part 131 may be provided on the lower wall of the second housing 130. The sterilizing lamp mounting part 131 may be provided on a central portion of the lower wall of the second housing 130. The central portion of the lower wall of the second housing 130 may be a position on basis of a flow direction of air that is introduced into the inside of the housing 110. Each of opposite ends of the sterilizing lamp 140 having a cylindrical shape may be mounted on the sterilizing lamp mounting part 131.

The second housing 130 may include a deodorizing filter mounting part 132 on which the deodorizing filter 150 is mounted. The deodorizing filter mounting part 132 may be provided on the lower wall of the second housing 130. The deodorizing filter mounting part 132 may be provided in front of the sterilizing lamp mounting part 131.

The second housing 130 may include a deodorizing lamp mounting part 133 on which a deodorizing lamp 160 is mounted. The deodorizing lamp mounting part 133 may be provided on the lower wall of the second housing 130. The deodorizing lamp mounting part 133 may be provided in front of the deodorizing filter mounting part 132. That is, the deodorizing lamp mounting part 133 may be provided on an opposite side of the sterilizing lamp mounting part 131 with respect to the deodorizing filter mounting part 132 as a center.

Accordingly, the inlet 111, the sterilizing filter mounting part 131, the deodorizing filter mounting part 132, and the deodorizing lamp mounting part 133, which are provided in the housing 110, may be arranged sequentially along the flow direction of air that is introduced into the inside of the housing 110. In other words, the inlet 111, the sterilizing lamp 140, the deodorizing filter 150, and the deodorizing lamp 160 may be arranged sequentially along the flow direction of air that is introduced into the inside of the housing 110.

The second housing 130 may include a deodorizing filter rib 134 provided on each of left and right sides of the deodorizing filter 150. That is, the deodorizing filter rib 134 may be provided on each of left and right sides of the deodorizing filter mounting part 132. The deodorizing filter rib 134 may prevent ultraviolet rays radiated from the sterilizing lamp 140 from leaking out through the discharge port 170. That is, the deodorizing filter rib 134 may prevent ultraviolet rays radiated from the sterilizing lamp 140 from leaking into the refrigerating chamber 22 (see FIG. 2) through the discharge port 170.

The deodorizing filter rib 134 may include a first deodorizing filter rib 135 provided on the left side of the deodorizing filter 150. That is, the first deodorizing filter rib 135 may be provided on a left side of the deodorizing filter mounting part 132. In this case, the left side of the deodorizing filter 150 may be referred by a basis on looking at the housing 110 from the front of the housing 110.

The deodorizing filter rib 134 may include a second deodorizing filter rib 136 provided on the right side of the deodorizing filter 150. That is, the second deodorizing filter rib 136 may be provided on a right side of the deodorizing filter mounting part 132.

A length D1 between the left end of the first deodorizing filter rib 135 and the right end of the second deodorizing filter rib 136 may be provided to be longer than a length D2 of the sterilizing lamp 140. Because the length D1 between the left end of the first deodorizing filter rib 135 and the right end of the second deodorizing filter rib 136 is provided to be longer than the length D2 of the sterilizing lamp 140, ultraviolet rays radiated from the sterilizing lamp 140 may be prevented from leaking into the refrigerating chamber 22 (see FIG. 2) through the discharge port 170.

The second housing 130 may include a deodorizing lamp rib 137 provided in front of the deodorizing lamp 160. That is, the deodorizing lamp rib 137 may be provided in front of the deodorizing lamp mounting part 133. The deodorizing lamp rib 137 may prevent ultraviolet rays radiated from the sterilizing lamp 140 from leaking out of the outside through the discharge port 170. That is, the deodorizing lamp rib 137 may prevent ultraviolet rays radiated from the sterilizing lamp 140 from leaking into the refrigerating chamber 22 (see FIG. 2) through the discharge port 170. Also, the deodorizing lamp rib 137 may prevent ultraviolet rays radiated from the deodorizing lamp 160 from leaking into the refrigerating chamber 22 (see FIG. 2) through the discharge port 170. A length D3 of the deodorizing lamp rib 137 may be provided to be longer than a length D4 of the deodorizing filter 150.

The deodorizing filter rib 134 and the deodorizing lamp rib 137 may guide air, which has been introduced into the housing 110 and sterilized and deodorized, to be smoothly discharged to the discharge port 170. That is, the air introduced into the housing 110 and sterilized and deodorized may be guided through a space between the deodorizing filter rib 134 and the deodorizing lamp rib 137 to be smoothly discharged to the discharge port 170.

The sterilization and deodorization device 100 may include the sterilizing lamp 140. The sterilizing lamp 140 may be a UVC (Ultraviolet C) lamp radiating ultraviolet rays. The sterilizing lamp 140 may be provided to have a cylindrical shape. Because the sterilizing lamp 140 is provided to have a cylindrical shape, the ultraviolet rays radiated from the sterilizing lamp 140 may be radiated in a 360-degree direction. The sterilizing lamp 140 may be mounted on the sterilizing lamp mounting part 131 provided in the second housing 130. Each of the opposite ends of the sterilizing lamp 140 may be mounted on the sterilizing lamp mounting part 131. The sterilizing lamp 140 may sterilize air introduced into the inlet 111 by radiating ultraviolet rays. The air sterilized by ultraviolet rays radiated from the sterilizing lamp 140 may be discharged into the refrigerating chamber 22 (see FIG. 2) through the discharge port 170.

The sterilizing lamp 140 may be provided between the inlet 111 and the deodorizing filter 150. Because the sterilizing lamp 140 is provided between the inlet 111 and the deodorizing filter 150, a sterilization area S in which air introduced into the inlet 111 is sterilized by ultraviolet rays radiated from the sterilizing lamp 140 may be formed in a section between the inlet 111 and the deodorizing filter 150. That is, the air introduced into the inlet 111 may be sterilized while flowing through the sterilization area S formed in the section between the inlet 111 and the deodorizing filter 150, and then discharged to the discharge port 170. As described above, because the sterilizing lamp 140, which is capable of radiating ultraviolet rays in the 360-degree direction, is disposed in a central portion of the housing 110, the sterilization area S in which air is sterilized may be secured to the maximum. Additionally, because the sterilizing lamp 140, which is capable of radiating ultraviolet rays in the 360-degree direction, is disposed in the central portion of the housing 110, the sterilization time to sterilize air may be secured to the maximum.

The sterilization area S may include a first sterilization area S1 formed in the section between the inlet 111 and the sterilizing lamp 140. The sterilization area S may include a second sterilization area S2 formed in a section between the sterilizing lamp 140 and the deodorizing filter 150.

A distance L1 between the inlet 111 and the sterilizing lamp 140, which is the first sterilization area S1, may be provided to be longer than a distance L2 between the sterilizing lamp 140 and the deodorizing filter 150, which is the second sterilization area S2. When the distance L1 between the inlet 111 and the sterilizing lamp 140, which is the first sterilization area S1, is longer than the distance L2 between the sterilizing lamp 140 and the deodorizing filter 150, which is the second sterilization area S2, not only may the sterilization area S be maximized, but the sterilizing lamp 140 and the deodorizing filter 150 may be positioned within a certain distance. When the sterilizing lamp 140 and the deodorizing filter 150 are positioned within a certain distance, the deodorizing filter 150 may be activated by ultraviolet rays radiated from the sterilizing lamp 140. When the deodorizing filter 150 is activated by ultraviolet rays radiated from the sterilizing lamp 140, air introduced into the inlet 111 may be deodorized by the deodorizing filter 150 and then discharged into the refrigerating chamber 22 (see FIG. 2) through the discharge port 170. As described above, because sterilization and/or deodorization of air is possible through the sterilizing lamp 140 which is a single lamp, the efficiency of power consumption may be increased. Additionally, the overall volume of the sterilization and deodorization device 100 may be minimized.

The sterilization and deodorization device 100 may include the deodorizing filter 150. The deodorizing filter 150 may be mounted on the deodorizing filter mounting part 132 provided in the second housing 130. The deodorizing filter 150 may be disposed in front of the sterilizing lamp 140. That is, the deodorizing filter 150 may be disposed such that the sterilizing lamp 140 is positioned between the inlet 111 and the deodorizing filter 150. The deodorizing filter 150 may be activated by ultraviolet rays radiated from the sterilizing lamp 140. The deodorizing filter 150 activated by ultraviolet rays radiated from the sterilizing lamp 140 may deodorize air introduced into the inlet 111. The air deodorized by the deodorizing filter 150 may be discharged into the refrigerating chamber 22 (see FIG. 2) through the discharge port 170.

The sterilization and deodorization device 100 may include the deodorizing lamp 160. The deodorizing lamp 160 may be mounted on the deodorizing lamp mounting part 133 provided in the second housing 130. The deodorizing lamp 160 may be disposed in front of the deodorizing filter 150. That is, the deodorizing lamp 160 may be provided on an opposite side of the sterilizing lamp 140 with respect to the deodorizing filter 150 as a center. Accordingly, the inlet 111, the sterilizing lamp 140, the deodorizing filter 150, and the deodorizing lamp 160 may be arranged sequentially along a flow direction of air that is discharged from the cold air duct discharge port 61 of the cold air duct 60 and introduced into the sterilization and deodorization device 100.

The deodorizing lamp 160 may selectively activate the deodorizing filter 150. When it is necessary to sterilize and deodorize air inside the refrigerating chamber 22 by detecting a level of contamination of the air inside the refrigerating chamber 22 or opening and closing of the refrigerating chamber door 31, the sterilizing lamp 140 may be turned on for sterilization and deodorization operations. When the sterilizing lamp 140 is turned on and ultraviolet rays are radiated from the sterilizing lamp 140, air introduced into the inlet 111 may be sterilized. Additionally, the deodorizing filter 150 may be activated by the ultraviolet rays radiated from the sterilizing lamp 140, so that the air introduced into the inlet 111 may be deodorized. The air sterilized and deodorized by the sterilizing lamp 140 and the deodorizing filter 150 may be discharged to the discharge port 170 (see FIGS. 1 and 2).

When the level of contamination inside the refrigerating chamber 22 and an accumulated operation time of sterilization/deodorization operation are determined and a required deodorization time is determined to have elapsed, the sterilizing lamp 140 may be turned off and the deodorizing lamp 160 may be turned on. That is, as the sterilizing lamp 140 turns off, sterilization of air is stopped, and as the deodorizing lamp 160 turns on, the deodorizing filter 150 is activated, so that deodorization of air by the deodorizing filter 150 may be continuously maintained.

When the level of contamination inside the refrigerating chamber 22 and the accumulated operation time of sterilization/deodorization operation are determined and the required deodorization time is determined to have elapsed, the deodorizing lamp 160 may be turned off.

The sterilization and deodorization device 100 may include the discharge port 170. The discharge port 170 may discharge air that has been introduced into the inlet 111 and sterilized and deodorized by the sterilizing lamp 140 and/or the deodorizing filter 150 into the refrigerating chamber 22 (see FIG. 2). The discharge port 170 may allow air, which has been introduced into the inlet 111 and sterilized and deodorized by the sterilizing lamp 140 and/or the deodorizing filter 150, to be discharged into the refrigerating chamber 22 (see FIG. 2). The discharge port 170 may be provided in the second housing 130. The discharge port 170 may be provided between the deodorizing filter 150 and the deodorizing lamp 160. Accordingly, the inlet 111, the sterilizing lamp 140, the deodorizing filter 150, the discharge port 170, and the deodorizing lamp 160 may be arranged sequentially along the flow direction of air that is discharged from the cold air duct discharge port 61 of the cold air duct 60 and introduced into the sterilization and deodorization device 100. As described above, because the inlet 111, the sterilizing lamp 140, the deodorizing filter 150, the discharge port 170, and the deodorizing lamp 160 are implemented along a straight flow path, loss of air flow may be minimized while air is being sterilized and/or deodorized.

The discharge port 170 may include a first discharge port 171 formed in a hole shape between the deodorizing filter 150 and the deodorizing lamp 160. The first discharge port 171 may be formed on the lower wall of the second housing 130. Air introduced into the inlet 111 and sterilized and deodorized by the sterilizing lamp 140 and/or the deodorizing filter 150 may be discharged into the refrigerating chamber 22 (see FIG. 2) through the first discharge port 171.

The discharge port 170 may include a second discharge port 173 formed on each of left and right sides of the first discharge port 171 by the deodorizing filter rib 134 and the deodorizing lamp rib 137. Air introduced into the inlet 111 and sterilized and deodorized by the sterilizing lamp 140 and/or the deodorizing filter 150 may be guided by the deodorizing filter rib 134 and the deodorizing lamp rib 137 to be discharged into the refrigerating chamber 22 (see FIG. 2) through the second discharge port 173.

The sterilization and deodorization device 100 may include a thermal insulating tape 180 attached to a wall surface of the housing 110. The thermal insulating tape 180 may be attached to the wall surface of the housing 110 adjacent to the refrigerating chamber 22 (see FIG. 2). The thermal insulating tape 180 may be attached to the lower wall of the inside of the second housing 130 adjacent to the refrigerating chamber 22 (see FIG. 2). The thermal insulating tape 180 may dissipate heat generated from the sterilizing lamp 140.

The thermal insulating tape 180 may include a reflective layer 181 reflecting ultraviolet rays radiated from the sterilizing lamp 140. The reflective layer 181 may be formed of a metallic material. The reflective layer 181 may prevent ultraviolet rays radiated from the sterilizing lamp 140 from directly reaching a surface of the housing 110 by reflecting the ultraviolet rays radiated from the sterilizing lamp 140. In particular, because when the housing 110 is formed as an injection molded product, ultraviolet rays radiated from the sterilizing lamp 140 are prevented from directly reaching the surface of the housing 110 by the reflective layer 181, photodegradation of the housing 110 may be prevented. Additionally, as ultraviolet rays radiated from the sterilizing lamp 140 are reflected by the reflective layer 181, a re-sterilization effect of air may be created.

The thermal insulating tape 180 may include a thermal insulating layer 183 preventing heat generated from the sterilizing lamp 140 from being transferred to the refrigerating chamber 22. The thermal insulating layer 183 may prevent heat generated when ultraviolet rays are radiated from the sterilizing lamp 140 from being transferred to the refrigerating chamber 22 and increasing a temperature inside the refrigerating chamber 22.

As described above, as the thermal insulating tape 180 is attached to the lower wall of the inside of the second housing 130 adjacent to the refrigerating chamber 22 (see FIG. 2), dew may be prevented from forming on a surface of the second housing 130 adjacent to the refrigerating chamber 22 (see FIG. 2). Additionally, as the thermal insulating tape 180 is attached to the lower wall of the inside of the second housing 130 adjacent to the refrigerating chamber 22 (see FIG. 2), loss of output of the sterilizing lamp 140 may be prevented. Additionally, as the thermal insulating tape 180 is attached to the lower wall of the inside of the second housing 130 adjacent to the refrigerating chamber 22 (see FIG. 2), the life of the sterilizing lamp 140 may be extended.

The thermal insulating tape 180 may be provided such that the reflective layer 181 is positioned over the thermal insulating layer 183.

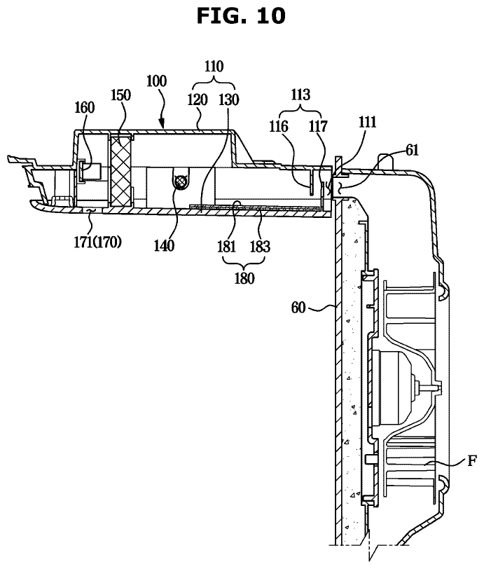

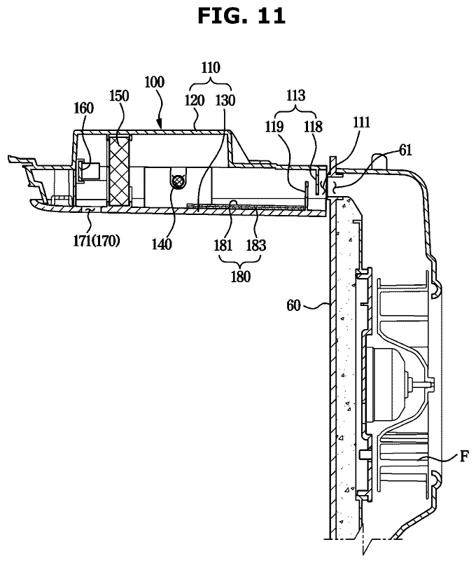

FIG. 10 is a cross-sectional view illustrating that a first blocking rib provided in a portion adjacent to an inlet of the sterilization and deodorization device is formed further in front than a second blocking rib according to an embodiment. FIG. 11 is a cross-sectional view illustrating that a second blocking rib provided in a portion adjacent to an inlet of the sterilization and deodorization device is formed further in front than a first blocking rib according to an embodiment.

As illustrated in FIG. 10, the housing 110 may include the blocking rib 113 preventing ultraviolet rays radiated from the sterilizing lamp 140 from leaking out of the housing 110 through the inlet 111. Air introduced into the housing 110 may be sterilized by ultraviolet rays radiated from the sterilizing lamp 140 mounted inside the housing 110. The ultraviolet rays radiated from the sterilizing lamp 140 may be prevented from leaking into the cold air duct 60 through the inlet 111 by the blocking rib 113.

The blocking rib 113 may be provided in each of the first housing 120 and the second housing 130 adjacent to the inlet 111. The blocking rib 113 may include a first blocking rib 116 formed in the first housing 120. The first blocking rib 116 may be formed to extend downward from the upper wall of the first housing 120.

The blocking rib 113 may include a second blocking rib 117 formed in the second housing 130. The second blocking rib 117 may be formed to extend upward from the lower wall of the second housing 130.

The first blocking rib 116 and the second blocking rib 117 may be formed at different positions on basis of the flow direction of air that is introduced into the inlet 111. The first blocking rib 116 may be formed further in front than the second blocking rib 117. That is, the second blocking rib 117 may be formed closer to the inlet 111 than the first blocking rib 116. Because the first blocking rib 116 and the second blocking rib 117 are formed at different positions on basis of the flow direction of air that is introduced into the inlet 111, the sum of lengths of the first blocking rib 116 and the second blocking rib 117 may be provided to be longer than a height of the inlet 111. Therefore, ultraviolet rays radiated from the sterilizing lamp 140 may be completely blocked from leaking into the cold air duct 60 through the inlet 111 by the first blocking rib 116 and the second blocking rib 117.

As illustrated in FIG. 11, the housing 110 may include the blocking rib 113 preventing ultraviolet rays radiated from the sterilizing lamp 140 from leaking out of the housing 110 through the inlet 111. Air introduced into the housing 110 may be sterilized by ultraviolet rays radiated from the sterilizing lamp 140 mounted inside the housing 110. The ultraviolet rays radiated from the sterilizing lamp 140 may be prevented from leaking into the cold air duct 60 through the inlet 111 by the blocking rib 113.

The blocking rib 113 may be provided in each of the first housing 120 and the second housing 130 adjacent to the inlet 111. The blocking rib 113 may include a first blocking rib 118 formed in the first housing 120. The first blocking rib 118 may be formed to extend downward from the upper wall of the first housing 120.

The blocking rib 113 may include a second blocking rib 119 formed in the second housing 130. The second blocking rib 119 may be formed to extend upward from the lower wall of the second housing 130.

The first blocking rib 118 and the second blocking rib 119 may be formed at different positions on basis of the flow direction of air that is introduced into the inlet 111. The second blocking rib 119 may be formed further in front than the first blocking rib 118. That is, the first blocking rib 118 may be formed closer to the inlet 111 than the second blocking rib 119. Because the first blocking rib 118 and the second blocking rib 119 are formed at different positions on basis of the flow direction of air that is introduced into the inlet 111, the sum of lengths of the first blocking rib 118 and the second blocking rib 119 may be provided to be longer than the height of the inlet 111. Therefore, ultraviolet rays radiated from the sterilizing lamp 140 may be completely blocked from leaking into the cold air duct 60 through the inlet 111 by the first blocking rib 118 and the second blocking rib 119.

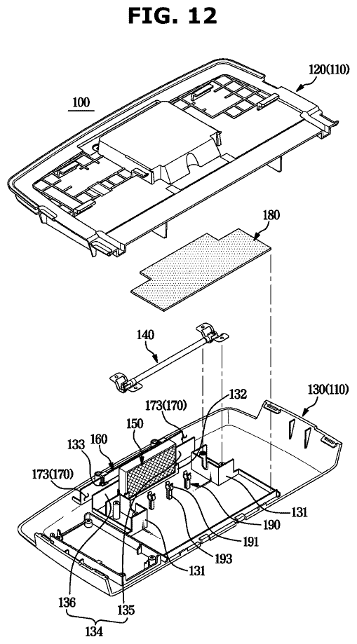

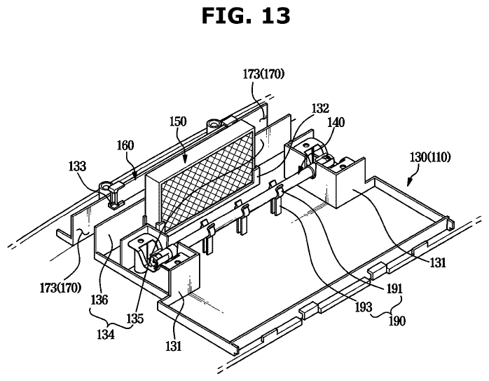

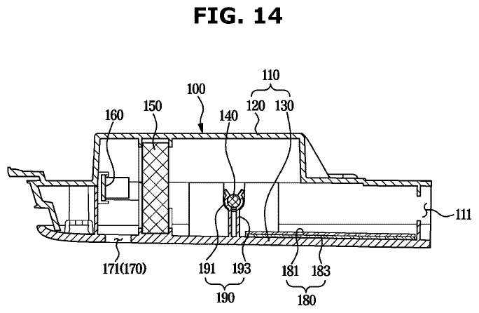

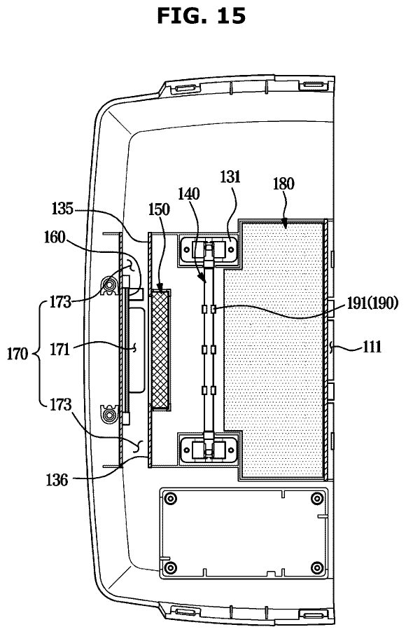

FIG. 12 is a view illustrating a state in which the sterilization and deodorization device including a heat dissipation support supporting the sterilizing lamp is exploded according to an embodiment. FIG. 13 is a view illustrating that the heat dissipation support supports the sterilizing lamp according to an embodiment. FIG. 14 is a schematic cross-sectional view of the sterilization and deodorization device including the heat dissipation support supporting the sterilizing lamp according to an embodiment. FIG. 15 is a plan view illustrating that the sterilizing lamp, the deodorizing filter, and the deodorizing lamp are mounted, the heat dissipation support supports the sterilizing lamp, and the thermal insulating tape is attached, in the inside of the second housing according to an embodiment.

As illustrated in FIGS. 12 to 15, the sterilization and deodorization device 100 may include a heat dissipation support 190 supporting the sterilizing lamp 140 formed in a cylindrical shape. The heat dissipation support 190 may be formed of a material capable of transferring heat generated from the sterilizing lamp 140. The heat dissipation support 190 may be formed of a metallic material. The heat dissipation support 190 may be provided in plurality. The heat dissipation support 190 may be provided to be in contact with the sterilizing lamp 140 to dissipate heat generated from the sterilizing lamp 140.

The heat dissipation support 190 may include a contact part 191 in contact with the sterilizing lamp 140. The contact part 191 may be provided to surround a portion of the sterilizing lamp 140.

The heat dissipation support 190 may include a support part 193 connected to the contact part 191. The support part 193 may have one end connected to the contact part 191 and the other end fixed to the wall surface of the housing 110 adjacent to the refrigerating chamber 22 to support the sterilizing lamp 140. The support part 193 may be provided such that at least a portion thereof is in contact with the wall surface of the housing 110 adjacent to the refrigerating chamber 22. Herein, the housing 110 adjacent to the refrigerating chamber 22 may be the second housing 130. Accordingly, the support part 193 may be provided such that at least a portion thereof is in contact with the lower wall of the second housing 130 (see FIG. 2).

As described above, as the heat dissipation support 190 formed of a metallic material is fixed to the lower wall of the inside of the second housing 130 adjacent to the refrigerating chamber 22, dew may be prevented from forming on the surface of the second housing 130 adjacent to the refrigerating chamber 22. Additionally, as the heat dissipation support 190 formed of a metallic material is fixed to the lower wall of the inside of the second housing 130 adjacent to the refrigerating chamber 22, the loss of output of the sterilizing lamp 140 may be prevented. Additionally, as the heat dissipation support 190 formed of a metallic material is fixed to the lower wall of the inside of the second housing 130 adjacent to the refrigerating chamber 22, the life of the sterilizing lamp 140 may be extended (see FIG. 2).

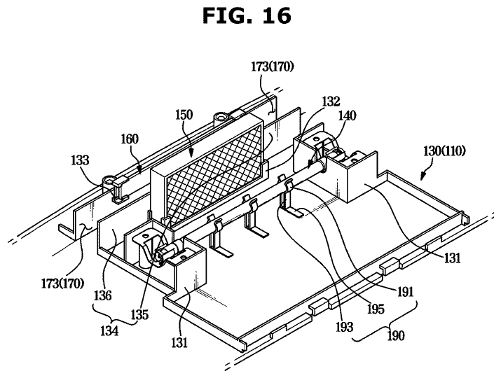

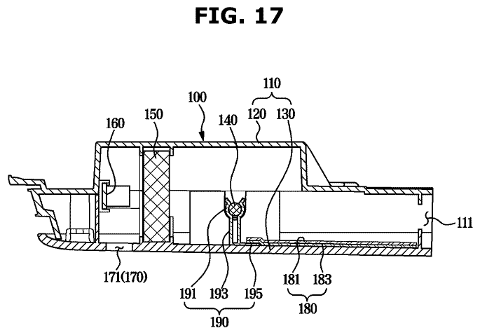

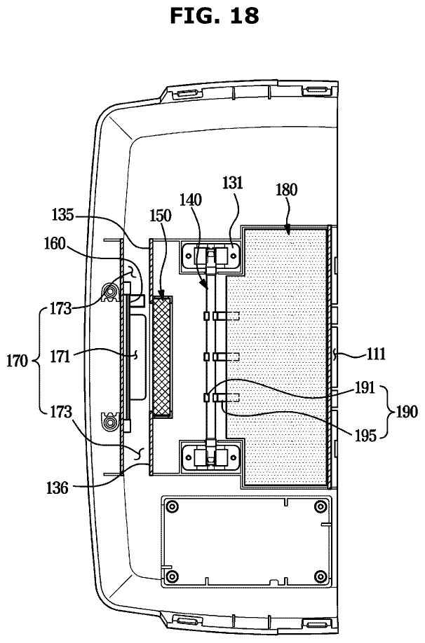

FIG. 16 is a view illustrating that the heat dissipation support supporting the sterilizing lamp includes an extension part extending from a support part according to an embodiment. FIG. 17 is a schematic cross-sectional view of the sterilization and deodorization device in which the heat dissipation support includes the extension part according to an embodiment. FIG. 18 is a plan view illustrating that the sterilizing lamp, the deodorizing filter, and the deodorizing lamp are mounted, the heat dissipation support including the extension part supports the sterilizing lamp, and the thermal insulating tape is attached, in the inside of the second housing according to an embodiment.

As illustrated in FIGS. 16 to 18, the heat dissipation support 190 may include an extension part 195 extending along the wall surface of the housing 110 from the support part 193. The extension part 195 may extend along a surface of the lower wall of the second housing 130. The extension part 195 may extend in a direction parallel to a flow direction of air that is introduced into the sterilization and deodorization device 100. That is, the extension part 195 may be extended in a direction parallel to the flow direction of air that is introduced into the sterilization and deodorization device 100 through the inlet 111. The drawings illustrate that the extension part 195 extends from the support part 193 to a length similar to a length of the support part 193, but the present disclosure is not limited thereto. That is, the extension part 195 may be provided to extend from the support part 193 to the inlet 111. The extension part 195 may be provided to have the same width as the support part 193.

The thermal insulating tape 180 may be attached to the wall surface of the housing 110 to cover an upper portion of the extension part 195. The thermal insulating tape 180 may be attached to the wall surface of the housing 110 to cover a portion of the upper portion of the extension part 195. That is, the thermal insulating tape 180 attached to the lower wall of the inside of the second housing 130 may be attached to the lower wall of the inside of the second housing 130 to cover a portion of the upper portion of the extension part 195. The drawings illustrate that the thermal insulating tape 180 is attached to the lower wall of the inside of the second housing 130 to cover a portion of the upper portion of the extension part 195, but the present disclosure is not limited thereto. That is, the thermal insulating tape 180 may be attached to the lower wall of the inside of the second housing 130 to cover the entire upper portion of the extension part 195.

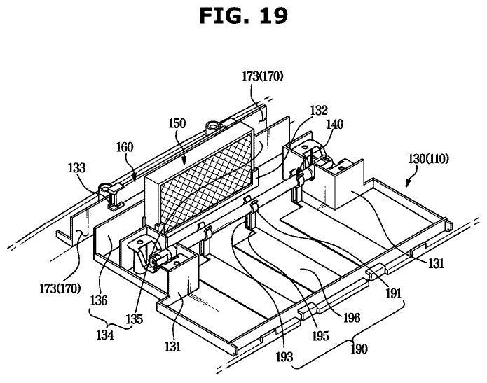

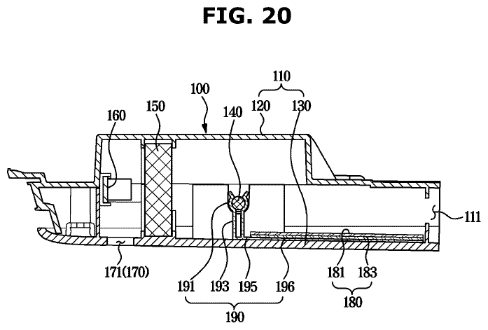

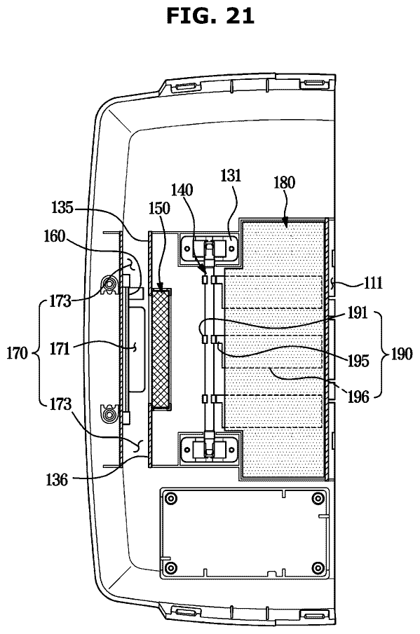

FIG. 19 is a view illustrating that the heat dissipation support includes an expansion part having a width wider than a width of the support part according to an embodiment. FIG. 20 is a schematic cross-sectional view of the sterilization and deodorization device in which the heat dissipation support includes the expansion part according to an embodiment. FIG. 21 is a plan view illustrating that the sterilizing lamp, the deodorizing filter, and the deodorizing lamp are mounted, the heat dissipation support including the expansion part supports the sterilizing lamp, and the thermal insulating tape is attached, in the inside of the second housing according to an embodiment.

As illustrated in FIGS. 19 to 21, the extension part 195 of the heat dissipation support 190 may include an expansion part 196 provided to have a width wider than the width of the support part 193. The expansion part 196, like the extension part 195, may extend along the surface of the lower wall of the second housing 130. The expansion part 196 may extend in a direction parallel to the flow direction of air that is introduced into the sterilization and deodorization device 100. That is, the expansion part 196 may extend in a direction parallel to the flow direction of air that is introduced into the sterilization and deodorization device 100 through the inlet 111. The drawings illustrate that the expansion part 196 extends to a position adjacent to the inlet 111, but the present disclosure is not limited thereto.

The thermal insulating tape 180 may be attached to the wall surface of the housing 110 to cover an upper portion of the expansion part 196. The thermal insulating tape 180 may be attached to the wall surface of the housing 110 to cover a portion of the upper portion of the expansion part 196. That is, the thermal insulating tape 180 attached to the lower wall of the inside of the second housing 130 may be attached to the lower wall of the inside of the second housing 130 to cover a portion of the upper portion of the expansion part 196. The drawings illustrate that the thermal insulating tape 180 is attached to the lower wall of the inside of the second housing 130 to cover a portion of the upper portion of the expansion part 196, but the present disclosure is not limited thereto. That is, the thermal insulating tape 180 may be attached to the lower wall of the inside of the second housing 130 to cover the entire upper portion of the expansion part 196.

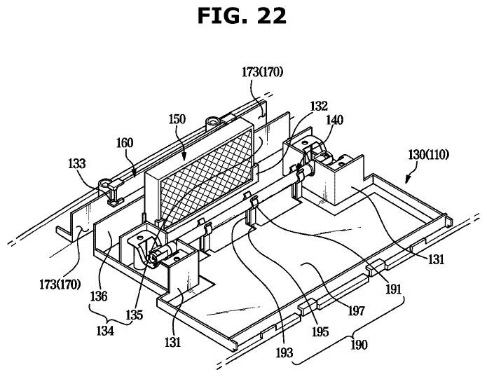

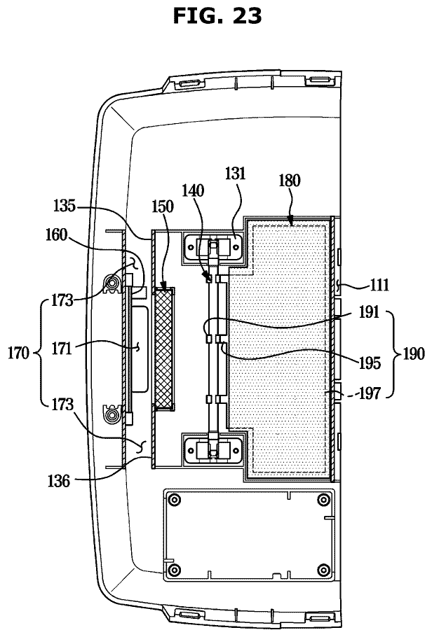

FIG. 22 is a view illustrating that expansion parts of the heat dissipation support are connected to each other in a direction perpendicular to a flow direction of air according to an embodiment. FIG. 23 is a plan view illustrating that the sterilizing lamp, the deodorizing filter, and the deodorizing lamp are mounted, the heat dissipation support including the expansion parts connected to each other in a direction perpendicular to the flow direction of air supports the sterilizing lamp, and the thermal insulating tape is attached, in the inside of the second housing according to an embodiment.

As illustrated in FIGS. 22 and 23, the extension part 195 of the heat dissipation support 190 may include an expansion part 197 provided to have a width wider than the width of the support part 193. The expansion part 197 may be provided to be connected to each other in a direction perpendicular to the flow direction of air that is introduced into the sterilization and deodorization device 100. That is, the expansion part 197 may be provided to be connected to each other in a direction perpendicular to the flow direction of air that is introduced into the sterilization and deodorization device 100 through the inlet 111. The expansion part 197, like the extension part 195, may extend along the surface of the lower wall of the second housing 130. The expansion part 197 may extend in a direction parallel to the flow direction of air that is introduced into the sterilization and deodorization device 100. That is, the expansion part 197 may extend in a direction parallel to the flow direction of air that is introduced into the sterilization and deodorization device 100 through the inlet 111. The drawings illustrate that the expansion part 197 extends to a position adjacent to the inlet 111, but the present disclosure is not limited thereto.

A refrigerator according to an embodiment of the present disclosure may include a cabinet 10, a storage compartment 20 provided inside the cabinet, a cold air duct 60 disposed at the rear of the storage compartment, and a sterilization and deodorization device 100 mounted inside the storage compartment and configured to introduce air discharged from the cold air duct, sterilize and deodorize the air, and then discharge the air into the storage compartment. The sterilization and deodorization device may include a housing 110 mounted in the storage compartment, a deodorizing filter 150 provided to deodorize air introduced into the housing, a sterilizing lamp 140 provided to sterilize air introduced into the housing and having a cylindrical shape, and a thermal insulating tape 180 attached to a wall surface of the housing adjacent to the storage compartment and including a reflective layer 181 reflecting ultraviolet rays radiated from the sterilizing lamp and an thermal insulating layer 183 preventing heat generated from the sterilizing lamp from being transferred to the storage compartment.

The thermal insulating tape may be attached to a wall surface of the inside of the housing adjacent to the storage compartment.

The reflective layer may be formed of a metallic material.

The sterilization and deodorization device may further include a heat dissipation support 190 provided to support the sterilizing lamp and dissipate heat generated from the sterilizing lamp.

The heat dissipation support may be provided in plurality and formed of a metallic material.

The heat dissipation support may include a contact part 191 in contact with the sterilizing lamp, and a support part 193 connected to the contact part and fixed to the wall surface of the housing adjacent to the storage compartment to support the sterilizing lamp.

The support part may be provided such that at least a portion thereof is in contact with the wall surface of the housing adjacent to the storage compartment.

The heat dissipation support may further include an extension part 195 extending along the wall surface of the housing from the support part.

The extension part may extend in a direction parallel to a flow direction of air that is introduced into the sterilization and deodorization device.

The extension part may be provided to have the same width as the support part.

The extension part may include an expansion part 196 or 197 provided to have a width wider than the width of the support part.

The expansion part 197 may be provided to be connected to each other in a direction perpendicular to the flow direction of air that is introduced into the sterilization and deodorization device.

The sterilization and deodorization device may further include an inlet 111 into which air discharged from the cold air duct is introduced.

The extension part may be provided to extend from the support part to the inlet.

The thermal insulating tape may be attached to the wall surface of the housing to cover an upper portion of the extension part.

A sterilization and deodorization device 100 according to an embodiment of the present disclosure, which introduces air discharged from a cold air duct 60 disposed at the rear of a storage compartment 20, sterilizes and deodorizes the air, and then discharges the air into the storage compartment, may include a housing 110 including a first housing 120 mounted on an upper wall of the storage compartment and a second housing 130 mounted on a lower portion of the first housing, an inlet 111 provided in the housing to allow air discharged from the cold air duct to be introduced into the housing, a deodorizing filter 150 provided to deodorize air introduced into the inlet, a sterilizing lamp 140 provided to sterilize air introduced into the inlet and having a cylindrical shape, a heat dissipation support 190 provided to support the sterilizing lamp and dissipate heat generated from the sterilizing lamp, and a thermal insulating tape 180 provided to be attached to a lower wall of the second housing and including a reflective layer 181 reflecting ultraviolet rays radiated from the sterilizing lamp and a thermal insulating layer 183 preventing heat generated from the sterilizing lamp from being transferring to the storage compartment.

The heat dissipation support may be formed of a metallic material and may be provided in plurality along a length direction of the sterilizing lamp.

The heat dissipation support may include a contact part 191 surrounding a portion of the sterilizing lamp to be in contact with the sterilizing lamp, and a support part 193 connected to the contact part to support the sterilizing lamp and provided such that at least a portion thereof is in contact with the lower wall of the second housing.

The heat dissipation support may further include an extension part 195 extending along the lower wall of the second housing in a direction parallel to a flow direction of air that is introduced into the inlet from the support part.

The thermal insulating tape may be attached to the lower wall of the second housing to cover an upper portion of the extension part.

According to the present disclosure, formation of dew on a wall surface of a housing adjacent to a storage compartment can be prevented.

Additionally, loss of output of a sterilizing lamp can be prevented.

Additionally, the life of the sterilizing lamp can be extended.

Additionally, photodegradation of a housing can be prevented.

Effects according to the present disclosure are not limited to the effects mentioned above, and other effects not mentioned will be clearly understood by those skilled in the art to which the present disclosure belongs from the fallowing description.

In describing the sterilization and deodorization device and the refrigerator having the same with reference to the accompanying drawings above, specific shapes and directions have been mainly described, but various modifications and changes may be made by a person skilled in the art, and such modifications and changes should be interpreted as being included in the scope of the present disclosure.

What is claimed is:

1. A refrigerator comprising:
a cabinet;
a storage compartment disposed inside the cabinet;
a cold air duct disposed at a rear of the storage compartment; and
a sterilization and deodorization device mounted inside the storage compartment, connected to the cold air duct, and configured to introduce air discharged from the cold air duct, sterilize and deodorize the introduced air, and discharge the sterilized and deodorized air into the storage compartment,
wherein the sterilization and deodorization device comprises:
a housing mounted in the storage compartment;
a deodorizing filter configured to deodorize the air introduced into the housing;
a sterilizing lamp configured to sterilize the air introduced into the housing, the sterilizing lamp having a cylindrical shape; and
a thermal insulating tape attached to a surface of the housing adjacent to the storage compartment, the thermal insulating tape comprising:
a reflective layer configured to reflect ultraviolet rays radiated from the sterilizing lamp; and
a thermal insulating layer configured to prevent heat generated from the sterilizing lamp from being transferred to the storage compartment.

2. The refrigerator according to claim 1, wherein
the housing includes a first housing mounted on an upper wall of the storage compartment and a second housing mounted on a lower portion of the first housing, and the surface of the housing is an inner surface of the second housing where the thermal insulating tape is attached to.

3. The refrigerator according to claim 1, wherein
the reflective layer is formed of a metallic material.

4. The refrigerator according to claim 1, wherein
the sterilization and deodorization device further comprises:
a heat dissipation support configured to support the sterilizing lamp and dissipate heat generated from the sterilizing lamp.

5. The refrigerator according to claim 4, wherein
the sterilization and deodorization device further comprises a plurality of the heat dissipation supports that is made of a metallic material.

6. The refrigerator according to claim 4, wherein
the heat dissipation support comprises:
a contact part configured to be in contact with the sterilizing lamp; and
a support part connected to the contact part and fixed to the surface of the housing adjacent to the storage compartment to support the sterilizing lamp.

7. The refrigerator according to claim 6, wherein
the support part is disposed such that at least a portion thereof is in contact with the surface of the housing adjacent to the storage compartment.

8. The refrigerator according to claim 6, wherein
the heat dissipation support further comprises:
an extension part extending along the surface of the housing from the support part.

9. The refrigerator according to claim 8, wherein
the extension part extends in a direction parallel to a flow direction of the air that is introduced into the sterilization and deodorization device.

10. The refrigerator according to claim 9, wherein
the extension part has a same width as the support part.

11. The refrigerator according to claim 10, wherein
the extension part comprises:
an expansion part has a width wider than the width of the support part.

12. The refrigerator according to claim 11, wherein
the expansion is to be connected to each other in a direction perpendicular to the flow direction of the air that is introduced into the sterilization and deodorization device.

13. The refrigerator according to claim 11, wherein
the sterilization and deodorization device further comprises:
an inlet into which the air discharged from the cold air duct is introduced.

14. The refrigerator according to claim 13, wherein
the extension part extends from the support part to the inlet.

15. The refrigerator according to claim 8, wherein
the thermal insulating tape is attached to the surface of the housing to cover an upper portion of the extension part.

16. The refrigerator according to claim 1, wherein
the sterilization and deodorization device further comprises an inlet into which the air discharged from the cold air duct is introduced, and
the housing includes a blocking rib to prevent the ultraviolet rays from leaking into the cold air duct through the inlet.

17. The refrigerator according to claim 16, wherein
the blocking rib includes a first blocking rib and a second blocking rib which is separated apart from the blocking rib in a flow direction of the air that is introduced into the sterilization and deodorization device.
